(19) United States
(12) Patent Application Publication
(10) Pub. No.: US 2022/0241204 A1
(43) Pub. Date: Aug. 4, 2022

(54) TOPICAL TIME RELEASE DELIVERY USING LAYERED BIOPOLYMER

(71) Applicant: DIOMICS CORPORATION, San Diego, CA (US)

(72) Inventors: Eric Mathur, San Diego, CA (US); Paul Wolff, Woodland Hills, CA (US); Jason Phillips, San Diego, CA (US)

(73) Assignee: DIOMICS CORPORATION, San Diego, CA (US)

(21) Appl. No.: 17/617,146

(22) PCT Filed: Jun. 5, 2020
(86) PCT No.: PCT/US2020/036496

§ 371 Date: Dec. 7, 2021

Related U.S. Application Data
(60) Provisional application No. 62/858,895, filed on Jun. 7, 2019.

(51) Int. Cl. A61K 9/16; A61K 9/00

(52) U.S. Cl. CPC A61K 9/1647; A61K 9/0014; A61K 9/0043

(57) ABSTRACT

The instant technology generally relates to a topical (e.g. transdermal) patch for delivery of an active agent. The patch comprises soluble, hydrophilic polycaprolactone (PCL) as a delivery substrate for an active agent of interest. Soluble, hydrophilic PCL can be formulated to adjust the degradation rate of the PCL, in order to deliver the active agent at a desired delivery rate.

### CROSS-REFERENCE TO RELATED APPLICATIONS

This application claims the benefit of U.S. Provisional Application No. 62/858,895 titled “TOPICAL TIME RELEASE DELIVERY USING LAYERED BIOPOLYMER” which was filed on Jun. 7, 2019 and is incorporated by reference in its entirety.

### FIELD OF THE INVENTION

This disclosure relates to the field of medical devices for drug delivery.

### BACKGROUND

Topical, transdermal and subdermal form fashions of the biopolymer matrix can be employed to administer active agents via absorption and co-resorption with the biopolymer through the skin, mucosal membranes and dermal layers of a subject, at controlled rates.

### SUMMARY

The instant technology generally relates to a topical, transdermal and subdermal biopolymer formats for delivery of biologically active agents. The biopolymer comprises soluble, hydrophilic polycaprolactone (PCL, an FDA approved bioresorbable polymer) as the delivery vehicle for active agents of interest. Soluble, hydrophilic PCL can be formulated in many form factors including transdermal patches, microbeads infused with active agents in topical creams or coupled to antigens for detection of viral infections. Our methods enable acceleration of PCL dissolution rates in order to deliver the active agents at desired rates.

In one aspect, a patch for application of an active agent to a patient is provided. In one aspect a topical patch for delivery of an active agent to a patient is provided. In one aspect a transdermal patch for the application of an active agent to the skin of a patient is provided.

In embodiments, the patch includes a backing layer. In embodiments, the patch includes a first active agent-containing layer, which may be adjacent to the backing layer. In embodiments, the first active agent-containing layer includes soluble, hydrophilic polycaprolactone and a first active agent. In embodiments, the first active agent-containing layer includes an adhesive.

In another aspect of this invention, the PCL polymer is prepared as microbeads ranging from 0.5-500 microns; the hydrophilic beads are imbibed with active agents and provided to the patients topically as skin creams.

In one embodiment, the biopolymer microbead consists of skin healing agents imbibed and bound electrostatically to the microbeads which are then added to a skin cream formulation for targeted delivery of the active agents to the sites of skin damage. For example wrinkles, or rhytides, or folds, ridges or creases in otherwise smooth skin surfaces. The microbead biopolymers may fill the rhytides and deliver stem cell derived biologicals directly to the sites of skin damage.

In another embodiment, the PCL microbeads are covalently coupled to reactive antigens derived from human or animal pathogens. The antigen-coupled PCL microbeads are then injected about 3 mm under the skin surface. The microbead polymer serves to tether and display the epitopes of the antigen which enables the individual's immune system to produce a localized immune response if the patient has circulating antibodies directed against the pathogen from which the antigen was derived. In this invention, the injected biopolymer serves as a real-time, long-term implantable diagnostic device for detection of previous infections to known pathogens.

In embodiments, the antigen is covalently coupled to the PCL via a peptide bond between exposed carbonyl groups created within the PCL microbead and amino groups of lysines present in the polypeptide chain of the antigen. The carbon:nitrogen bonds sequester the antigen, while the porous, microweb-nature of the polymer permit effective presentation of the antigen to the individual's immune response.

In recent results supporting these efforts, a successful animal trial was conducted which validated the concept of the polymer-antigen diagnostic medical device concept. The animal study utilized 12 egg-laying hens (courtesy of Hidden Villa Ranch, Calif.) previously immunized against avian infectious bronchitis virus (IBV). IBV is a gammacoronavirus and is a serious avian pathogen closely related to Covid-19, which is in the betacoronavirus family. Following chemical coupling of the IBV antigen (whole virus particles) to 3 mm×5 mm coupons of PCL with bovine serum albumin (BSA) was coupled to PCL as a non-immunoreactive control. The IBV antigen-bound PCL coupons were implanted directly onto muscle tissue of the left-wing webs of the 12 hens; control PCL coupons without bound antigen were implanted in the right-wing webs of the same birds. Initial skin surface reactivity and assessment of localized immune responses was evaluated at 24, 48, 72 and 96 hours after which time they were sacrificed, wing web implant sites surgically removed and stored in formalin for histological examination for the immune factors and components expected to be associated with localized immune responses. Visual results after 96 hours depict a dose dependent inflammation response present at the sites of all the IBV-Diomat PPD implantations, but no localized immune response present at the sites of the control implants.

In yet another embodiment, PCL is electrospun into microthin fibers woven into a gauze material and infused with exosomes, growth factors and cytokines derived from mesenchymal stem cell conditioned media and used for primary care for burn patient and wound healing.

In this embodiment, the ‘PCL gauze’ is imbibed with wound healing ointments and stem-cell derived biologicals targeted for wound healing and wrapped directly around burns and severe wounds with the PCL's rapid dissoluting and bioresorbtion eliminating the need to remove the bandage combined with the skin healing properties of the stem cell biologicals.

In embodiments, the soluble, hydrophilic polycaprolactone has been treated with a base having a pH greater than about 8, 9, 10, 11 or 12 and a neutralizing agent for increasing hydrophilicity. In embodiments, the base is at least one or a combination of NaOH, NaHCO₃, KOH, Na2C03, or Ca(OH)2. The dissolution rate of the polycaprolactone may increase during base treatment. In various embodiments, a higher pH of the base treatment results in a more rapid increase in the dissolution rate.

In embodiments, the first active agent-containing layer contains a co-polymer of polycaprolactone (e.g., soluble, hydrophilic polycaprolactone) and a second polymer. In embodiments, the second polymer is polylactic acid, acrylamide, polylactide, polyglycolide, polydioxanone, poly N-isopropylacrylamide, polyurethane, a polyester other than PCL, a polystyrene, or a polyvinylidene.

In embodiments, the patch contains a second active agent-containing layer. In embodiments, the second active agent-containing layer includes soluble, hydrophilic polycaprolactone. In embodiments, the second active agent-containing layer includes a co-polymer of polycaprolactone (e.g., soluble, hydrophilic polycaprolactone) and a third polymer. In embodiments, the third polymer is polylactic acid, acrylamide, polylactide, polyglycolide, polydioxanone, poly N-isopropylacrylamide, polyurethane, a polyester other than PCL, a polystyrene, or a polyvinylidene.

In embodiments, the second active agent-containing layer includes the first active agent. In embodiments, the second active agent-containing layer includes a second active agent.

In embodiments, the active agent-containing layer(s) dissolves over time after application to a subject. In embodiments, the active agent-containing layer(s) dissolves in about 30 hours to about one month. In embodiments, the first active agent-containing layer and the second active agent-containing layer have different dissolution rates. In embodiments, the first active agent-containing layer and the second active agent-containing layer have different active agent elution rates.

In embodiments, the patch includes a film layer over the first active agent-containing layer. In embodiments, the film comprises the first active agent, the second active agent, or a third active agent. In embodiments, the film dissolves over time after application to a subject. In embodiments, the film comprises polycaprolactone. In embodiments, the film comprises soluble, hydrophilic polycaprolactone. In embodiments, the film acts as a vapor barrier.

In embodiments, the first active agent-containing layer is porous. In embodiments, the second active agent-containing layer is porous.

In embodiments, the first active agent, second active agent, or third active agent comprises a nutraceutical. In embodiments, the first active agent, second active agent, or third active agent comprises a cannabinoid. In embodiments, the cannabinoid is cannabidiol.

In embodiments, the patch is for administration to a mucosal membrane of a patient. In embodiments, the patch is for administration to a wound.

An exemplary embodiment is a transdermal patch. The transdermal patch includes a first active agent-containing layer for application of an active agent to an area of skin of a patient and a backing layer adjacent to the first active agent-containing layer. The first active agent-containing layer includes soluble, hydrophilic polycaprolactone and a first active agent and has been treated with a base having a pH greater than 8 and a neutralizing agent for increasing hydrophilicity. The transdermal patch may further include a second active agent-containing layer. The first active agent-containing layer and the second active agent-containing layer may be configured to have different dissolution rates. The second active agent-containing layer may include a co-polymer of polycaprolactone and a third polymer where the third polymer is selected from polylactic acid, acrylamide, polylactide, polyglycolide, polydioxanone, poly N-isopropylacrylamide, polyurethane, a polyester other than polycaprolactone, a polystyrene, or a polyvinylidene. The transdermal patch may further include a film layer over the first active agent-containing layer. The film layer may be configured to dissolve over time after application to a subject. The film layer may act as a vapor barrier.

Anther general aspect is a topical patch. The topical patch includes a first active agent-containing layer and a backing layer adjacent to the first active agent-containing layer. The first active agent-containing layer includes soluble, hydrophilic polycaprolactone and a first active agent and has been treated with a based having a pH greater than 8 and a neutralizing agent for increasing hydrophilicity. The topical patch may be configured to be administered to a mucosal membrane or a wound of a patient. The topical patch may further include a second active agent-containing layer. The first active agent-containing layer and the second active agent-containing layer may be configured to have different dissolution rates. The second active agent-containing layer may include a co-polymer of polycaprolactone and a third polymer where the third polymer is selected from polylactic acid, acrylamide, polylactide, polyglycolide, polydioxanone, poly N-isopropylacrylamide, polyurethane, a polyester other than polycaprolactone, a polystyrene, or a polyvinylidene. The topical patch may further include a film layer over the first active agent-containing layer. The film layer may be configured to dissolve over time after application to a subject. The film layer may act as a vapor barrier.

An exemplary embodiment is a method for manufacturing a transdermal patch. The method includes forming a first layer of a polyester material with a thickness and treating the first layer with a base having a pH greater than 8 and a neutralizing agent for increasing hydrophilicity. The method includes adding an active agent into the first layer and layering a backing layer over the first layer. The polyester material may be polycaprolactone that is copolymerized with at least one copolymerizing agent selected from the group consisting of: polylactic acid, acrylamide, polylactide, polyglycolide, polydioxanone, poly N-isopropylacrylamide, polyurethane, a polyester other than PCL, a polystyrene, or a polyvinylidene. The method may further include controlling a rate of dissolution of the first layer. Controlling may include modifying a thickness of the first layer, modifying a time of treatment with the base, changing a molecular weight of the polyester material, and changing a percentage of weight per volume of polyester material. The method may further include forming a second layer comprising polyester material with a second thickness and adding an active agent into the second layer. The method may further include controlling a dissolution rate of the second layer by modifying the thickness of the first layer and controlling the dissolution rate of the second layer with a selection of a copolymerizing agent. The method may further include controlling the dissolution rate of the second layer by controlling an amount of the copolymerizing agent.

Anther general aspect is a topical treatment medium. The topical treatment medium includes a polyester material that has been treated with a based having a pH greater than 8 and a neutralizing agent for increasing hydrophilicity where the polyester material coupled to an active agent. The polyester material may include microbeads. The active agent may be a protein antigen that is covalently coupled to the microbeads. The protein antigen may be covalently coupled to the microbeads through a peptide bond. The protein antigen may be produced from a formation of a Schiff-Base. The polyester material may include thin fibers. The thin fibers may be woven into a mesh configured to bandage a wound where the active agent is a stem cell biological. The mesh may be configured to dissolve after being applied to the wound. The polyester material may be configured to be disposed on a backing.

An exemplary embodiment is a chemical composition. The chemical composition includes a polyester that is N substituted to form an imine. The imine may be coupled to an active agent. The imine may have multiple amino groups. Each of the multiple amino groups may be coupled to a separate active agent. The separate active agents may be identical. The separate active agents may have different chemical compositions. The polyester may be polycaprolactone. The polyester may be selected from a group consisting of polylactic acid, acrylamide, polylactide, polyglycolide, polydioxanone, poly N-isopropylacrylamide, polyurethane, poly(gamma-valerolactone), a polystyrene, or a polyvinylidene. The polyester may be a polyester other than polycaprolactone. At least one of the active agents may include a nutraceutical. At least one of the active agents may include cannabinoid. The cannabinoid may be cannabidiol.

Another general aspect is a transdermal patch. The transdermal patch includes one or more outer layers comprising, polycaprolactone that is base treated and exhibits hydrophilic properties and one or more inner layers comprising polycaprolactone that is base treated and exhibits hydrophilic properties. The polycaprolactone of the one or more outer layers is coupled to a first active agent and the polycaprolactone of the one or more inner layers coupled to a second active agent. The one or more outer layers may have a thickness of about 100-300 μm. The polycaprolactone of the one or more outer layers may have an average molecular weight of about 80,000 g/mol-120,000 g/mol. The polycaprolactone of the one or more outer layers may have a weight per volume concentration of about 5-8%. The one or more inner layers may have a thickness of about 10-50 μm. The polycaprolactone of the one or more inner layers may have an average molecular weight of about 20,000 g/mol-80,000 g/mol. The polycaprolactone of the one or more inner layers may have a weight per volume concentration of about 3-5%. At least one of the first active agent or the second active agent may be a nutraceutical. At least one of the first active agent or the second active agent may be a cosmeceutical. At least one of the first active agent or the second active agent may be a diagnostic monitoring agent. The microbeads may have a diameter of about 1-10 μm. The microbeads may be lyophilized. The active agent may be a mixture of at least one of stem cells, cytokines, and growth factors. The microbeads may be mixed in a skin cream. The microbeads mixed in the skin cream may be configured to be applied to skin of a subject to fill wrinkles. The active agent may be is a mixture of a convalescent antiserum, passive immune agent. The microbeads may be configured to be applied via intranasal aerosolization. The polycaprolactone may have a molecular weight of about 80,000 g/mol-120,000 g/mol. The polycaprolactone may have a weight per volume concentration of about 5-8%. The microbeads may have a diameter of about 1-100 μm. The active agent may be an antigen or a whole pathogen. The microbeads may be configured to be implanted into a subject via a 26-gauge syringe.

An exemplary embodiment is a treatment substance. The treatment substance includes microbeads comprising: polycaprolactone that is infused with an active agent. The polycaprolactone may have a molecular weight of about 20,000 g/mol-80,000 g/mol. The polycaprolactone may have a weight per volume concentration of about 3-5%. The microbeads may have a diameter of about 1-10 μm. The microbeads may be lyophilized. The active agent may be a mixture of at least one of stem cells, cytokines, and growth factors. The microbeads may be mixed in a skin cream. The microbeads may be mixed in the skin cream are configured to be applied to skin of a subject to fill wrinkles. The active agent may be a mixture of a convalescent antiserum, passive immune agent. The microbeads may be configured to be applied via intranasal aerosolization. The polycaprolactone may have a molecular weight of about 80,000 g/mol-120,000 g/mol. The polycaprolactone may have a weight per volume concentration of about 5-8%. The microbeads may have a diameter of about 1-100 μm. The active agent may be an antigen or a whole pathogen. The microbeads may be configured to be implanted into a subject via a 26-gauge syringe. Another general aspect is a treatment fabric. The treatment fabric includes microfibers comprising polycaprolactone that is coupled to an active agent where the microfibers are woven into a gauze that is configured to be applied to a subject. The polycaprolactone may have an average molecular weight of about 20,000 g/mol-80,000 g/mol. The polycaprolactone may have a weight per volume concentration of about 3-5%. A thickness of the microfibers may be about 0.5-10 μm. The active agent may be a stem cell biological. The gauze may be further configured to dissolve without being removed from the subject. The gauze may be fixed to an underside of a bandage. The bandage may be configured to be applied to a body part of the subject after the body part is tattooed.

### BRIEF DESCRIPTION OF THE DRAWINGS

FIG. 1 is an illustration of examples of various embodiments of topical patches.

FIG. 2A is an illustration of a chemical composition of a polycaprolactone molecule that is coupled to an active agent.

FIG. 2B is an illustration of a chemical composition of a polycaprolactone molecule with an array of potential active agents for which the molecule may be bound.

FIG. 3 is an illustration of a medical device with various polycaprolactone layers that may release an active agent into a subject.

FIG. 4 is a photograph of an embodiment of Diomat® film.

FIG. 5 is a photograph of an embodiment of Diomat® foam.

FIG. 6A is an electron microscopy image showing the microporous structure of the base-treated Diomat® foam.

FIG. 6B is an electron microscopy image showing the microporous structure of PCL microbeads.

FIG. 7A is a magnified photograph of an embodiment of polycaprolactone fibers that are produced from electrospinning.

FIG. 7B is a magnified photograph of an embodiment of polycaprolactone rods that may be bound in a microstructure.

FIG. 7C is a magnified photograph of polycaprolactone rods that are bound by polycaprolactone fibers in a microstructure.

FIG. 7D is a magnified photograph of a microstructure that is constructed from polycaprolactone rods that are bound by polycaprolactone fibers.

FIG. 8 is a reaction diagram of base-catalyzed hydrolysis of the ester linkages present in the backbone of poly caprolactone.

FIG. 9 is a reaction diagram of the coupling of surface-exposed carbonyl groups to create a Schiff base.

FIG. 10 is a magnified photograph of a hydrophobicity test of a water droplet on a wafer of poly caprolactone.

FIG. 11A is an image of a full Diomat® sheet sample S1.

FIG. 11B is an inverted brightfield microscopy image of Diomat® sample S1 using 4× objective.

FIG. 11C is an electron microscopy image of side-profile of Diomat® sample S1 using 1 mm (61×) resolution.

FIG. 11D is an electron microscopy image of side-profile of Diomat® sample S1 using 1 mm (503×) resolution.

FIG. 12A is an image of a full Diomat® sheet sample S2.

FIG. 12B is an inverted brightfield microscopy image of Diomat® sample S2 using 4× objective.

FIG. 12C is an electron microscopy image of side-profile of Diomat® using 0.5 mm (87×) resolution.

FIG. 12D is an electron microscopy image of side-profile of Diomat® using 0.5 mm (87×) resolution.

FIG. 13A is an image of a full Diomat® sheet sample S3.

FIG. 13B is an inverted brightfield microscopy image of Diomat® sample S3 using 4× objective.

FIG. 13C is an electron microscopy image of side-profile of Diomat® using 0.5 mm (87×) resolution.

FIG. 13D is an electron microscopy image of side-profile of Diomat® using 0.5 mm (87×) resolution.

FIG. 14A is an image of a full Diomat® sheet sample S4.

FIG. 14B is an inverted brightfield microscopy image of Diomat® sample S4 using 4× objective.

FIG. 14C is an electron microscopy image of side-profile of Diomat® using 0.5 mm (59×) resolution.

FIG. 14D is an electron microscopy image of side-profile of Diomat® using 0.5 mm (127×) resolution.

### DETAILED DESCRIPTION

After reading this description it will become apparent to one skilled in the art how to implement the invention in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

### Definitions

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “active agent” as used herein refers to any drug, pharmaceutical, nutraceutical, protein, collagen, biomaterial, etc. The active agents may be, for example, systemic or topical drugs. Individual active agents or mixtures thereof, if desired, can be employed. Any drug which passes through the skin or mucosa can be employed for internal administration in the device of the invention, so long as the drug will pass through the permeable adhesive layer or layers present and is stable within the patch. Suitable systemic drugs for administration by the patches described herein include, without limitation, psychoactive agents such as nicotine, caffeine, mesocarb, mefexamide, cannabinols such as THC and the like, cannabinoids such as CBD and the like, sedatives such as diazepam, mepiridine, uldazepam, tybamate, metaclazepam, tetrabarbitol and the like, antidepressants such as amitryptyline, imipramine desipramine, nialamide, melitracen, isocarboxazid, and the like, anticonvulsants such as phenobarbital, carbamazepine, methsuximide, 2-ethyl-2-phenylmalonamide (PEMA), phenytoin and the like, steroids such as progesterone, testosterone, pregnanediol, progestin, estradiol, analbolic steroids and the like, analgesics, including narcotic analgesics such as codeine, morphine, analorphine, demeral and the like, and analgesics such as acetaminophen, aspirin, alprazolam and the like, antimicrobial agents such as sulconazole, siccanin, silver sulfadiazine, bentiacide, and the like, tranquilizers such as meprobamate and the like, antineoplastic agents such as sulfosfamide, rufocromomycin and the like, and antibiotic agents such as tetracycline, penicillin, streptozcin and the like.

Active agent enhancers suitable for use in transdermal delivery patches are well-known and described, for example in U.S. Pat. No. 4,573,996, the disclosure of which is hereby incorporated herein by reference thereto. Active agent enhancers may promote administration of the active agent to a subject, for example by promoting the penetration of the active agent through the skin. The active agent enhancer may be incorporated into the patch in any layer, or in multiple layers.

By the phrase “modified PCL” is meant any PCL that has been treated or modified such that the hydrophilicity of the PCL is increased and/or such that one or more surface features of the PCL have been modified (e.g., chemical and/or physical modifications). Examples of surface features include texture (e.g., roughness, smoothness), holes, dimples, channels, protrusions and other irregularities. Any suitable treatment methods, including chemical or physical treatments, for increasing hydrophilicity and/or modifying surface features of PCL can be used. For example, PCL can be subjected to (treated with) a base (e.g. having a pH above 8). Non-limiting examples of bases include NaHCO3 and NaOH.

As used herein, the phrase “soluble and hydrophilic PCL” means polycaprolactone (PCL) that has been treated in some manner to make it absorb water and to increase its solubility (i.e., increase dissolution rate) when used in a patch.

As used herein, the term “copolymerized” refers to using two or more monomeric units to form a polymer with inclusion of both in some random (e.g., AABABBBAABAAABBBBA) or defined order (such as, e.g., AAABAAABAAAB or ABABABAB or ABAABAABAABAABAABA). For example, when referring to PCL that is copolymerized with at least one agent such as, e.g., L-lactic acid, the copolymer formed is a poly caprolactide called poly-L-lactic-co-£-caprolactone.

Polycaprolactone (PCL)

PCL is a monopolymer made by a ring-opening polymerization of epsilon caprolactone. Similar polymers are polylactide, polyglycolide or polydioxanone. PCL may be copolymerized with other esters such as polylactide, polyglycolide polydioxanone, poly(gamma-valerolactone), or poly (3 TolO-membered) lactone ring-containing compounds to alter properties. Polymers of acrylamide may also be used, such as poly N-isopropylacrylamide. In some embodiments, the PCL is copolymerized with a polystyrene or a polyvinylidene. Any suitable polystyrene can be used. Any suitable polyvinylidene can be used. Examples of polystyrenes that can be used include polystyrene, polystyrene sulfonate, carboxylated polystyrene, carboxyl are modified polystyrene, iodinated polystyrene, brominated polystyrene, chlorinated polystyrene, fluorinated polystyrene, lithium polystyryl modified iodinated polystyrene, iodinated polystyrene derivatives, polystyrene ionomers, polystyrene ion exchange resin, sodium polystyrene sulfonate, polystyrene sulfonate, chlorinated polystyrene derivatives, brominated polystyrene derivatives and derivatives thereof. Examples of polyvinylidene include polyvinylidine fluoride, polyvinylidine chloride, polyvinylidine bromide, polyvinylidine iodide, polyvinylidine acetate, polyvinylidine alcohol and derivatives thereof. Further examples of suitable agents for copolymerizing with PCL include polyvinylpyrrolidone, polyvinylpyrrolidone iodine, polyvinylpyrrolidone bromide, polyvinylpyrrolidone chloride, polyvinylpyrrolidone fluoride, polyethylene, iodinated polyethylene, brominated polyethylene, chlorinated polyethylene, fluorinated polyethylene, polyethylene terephthalate, polypropylene, iodinated polypropylene, brominated polypropylene, chlorinated polypropylene, fluorinated polypropylene and derivatives thereof.

Soluble, hydrophilic polycaprolactone as described herein can be made, for example, using the methods described in U.S. Pat. No. 9,359,600, which is incorporated herein by reference in its entirety. In embodiments, the soluble, hydrophilic polycaprolactone is DIOMAT®. DIOMAT® has been described, for example, in U.S. Pat. Nos. 9,708,600; 9,359,600; 8,759,075; 9,662,096; and 8,685,747; and U.S. Pub. Nos. 2016/0025603 and 2016/0047720, each of which is incorporated herein by reference in its entirety.

Hydrophobicity Overview

When an interface exists between a liquid and a solid, the angle between the surface of the liquid and the outline of the contact surface is described as the contact angle θ (lower case theta). The contact angle (wetting angle) is a measure of the wettability of a solid by a liquid. The wettability of biomaterials are important determinants of their function in vivo, as different proteins could get adsorbed upon implantation.

Soluble, hydrophilic PCL for use in the patches described herein may be in any form. In embodiments, the PCL is a film, for example. In embodiments, the PCL is a porous mesh or foam. The PCL-containing layer can incorporate/absorb the active agent. The active agent may be incorporated/absorbed in various ways. In various embodiments, the active agent may be covalently bonded to the PCL. In various embodiments, the active agent may be absorbed through pores of the PCL. In one instance, the active agent may be absorbed into pores of PCL that has been lyophilized, which may modify the internal structure of the PCL. In various embodiments, the active agent may be coupled to the PCL through electrostatic forces. In various embodiments, the active agent may be coupled to the PCL through a peptide bond. In various embodiments, the active agent may be coupled to the PCL through an ionic bond.

In various embodiments, a PCL-containing material may be treated to produce a micro-webbed structure in the PCL. In one example, the micro-webbed structure may be produced by lyophilizing the PCL. In various embodiments, the PCL that is modified to produce a micro-webbed structure, may sequester an active agent within the micro-webbed structure.

In various embodiments, a PCL-containing material may be treated under extreme hot or cold conditions. For example, the PCL-containing material may be treated to liquid nitrogen

In embodiments, the PCL-containing layer releases the active agent over time after administration of the patch to a subject. In embodiments, the PCL-containing layer dissolves or breaks down over time to release the active agent over time after administration of the patch to a subject. In embodiments, the elution/release rate of the active agent is dependent on the properties of the PCL. For example, a thicker PCL layer is expected to take a longer time to release the active agent (e.g., slower elution rate, and/or longer lifespan of the patch) than a thinner PCL layer. In embodiments, the PCL is combined with one or more additional polymers to form a co-polymer to adjust the elution/dissolution rate of the layer. For example, a co-polymer that dissolves more quickly than PCL alone after administration of the patch to a subject can be used. Alternatively, a co-polymer that dissolves more slowly than PCL alone after administration of the patch to a subject can be used. In embodiments, multiple PCL (or co-polymer) layers are used, each layer having a defined active agent elution rate (or defined PCL or co-polymer dissolution rate).

In embodiments, the active agent-containing layer(s) dissolves in about one day to about one month. Each active agent-containing layer can have a different dissolution rate than any other active-agent containing layer in the patch. In embodiments, the active agent-containing layer(s) dissolves in about 24 hours to about 72 hours. In embodiments, the active agent-containing layer(s) dissolves in about 30 hours to about 72 hours. In embodiments, the active agent-containing layer(s) dissolves in about 24 hours to about 4 weeks. In embodiments, the active agent-containing layer(s) dissolves in about 24 hours to about 3 weeks. In embodiments, the active agent-containing layer(s) dissolves in about 24 hours to about 2 weeks. In embodiments, the active agent-containing layer(s) dissolves in about 24 hours to about 1 week. In embodiments, the active agent-containing layer(s) dissolves in about 24 hours to about 6 days, 5 days, 4 days, 3 days, or 2 days. In embodiments, the active agent-containing layer(s) dissolves in about 30 hours to about 1 month. In embodiments, the active agent-containing layer(s) dissolves in about 30 hours to about 4 weeks. In embodiments, the active agent-containing layer(s) dissolves in about 30 hours to about 3 weeks. In embodiments, the active agent-containing layer(s) dissolves in about 30 hours to about 2 weeks. In embodiments, the active agent-containing layer(s) dissolves in about 30 hours to about 1 week. In embodiments, the active agent-containing layer(s) dissolves in about 30 hours to about 6 days, 5 days, 4 days, 3 days, or 2 days. In embodiments, the active agent-containing layer(s) dissolves in about 30 hours to about 1 month. In embodiments, the active agent-containing layer(s) dissolves in about 48 hours to about 4 weeks. In embodiments, the active agent-containing layer(s) dissolves in about 48 hours to about 3 weeks. In embodiments, the active agent-containing layer(s) dissolves in about 48 hours to about 2 weeks. In embodiments, the active agent-containing layer(s) dissolves in about 48 hours to about 1 week. In embodiments, the active agent-containing layer(s) dissolves in about 48 hours to about 6 days, 5 days, 4 days, or 3 days. In embodiments, the active agent-containing layer(s) dissolves in about 3 days to about 1 month. In embodiments, the active agent-containing layer(s) dissolves in about 3 days to about 4 weeks. In embodiments, the active agent-containing layer(s) dissolves in about 3 days to about 3 weeks. In embodiments, the active agent-containing layer(s) dissolves in about 3 days to about 2 weeks. In embodiments, the active agent-containing layer(s) dissolves in about 3 days to about 1 week. In embodiments, the active agent-containing layer(s) dissolves in about 3 days to about 6 days, 5 days, or 4 days. In embodiments, the active agent-containing layer(s) dissolves in about 4 days to about 1 month. In embodiments, the active agent-containing layer(s) dissolves in about 4 days to about 4 weeks. In embodiments, the active agent-containing layer(s) dissolves in about 4 days to about 3 weeks. In embodiments, the active agent-containing layer(s) dissolves in about 4 days to about 2 weeks. In embodiments, the active agent-containing layer(s) dissolves in about 4 days to about 1 week. In embodiments, the active agent-containing layer(s) dissolves in about 4 days to about 6 days, or 5 days. In embodiments, the active agent-containing layer(s) dissolves in about 5 days to about 1 month. In embodiments, the active agent-containing layer(s) dissolves in about 5 days to about 4 weeks. In embodiments, the active agent-containing layer(s) dissolves in about 5 days to about 3 weeks. In embodiments, the active agent-containing layer(s) dissolves in about 5 days to about 2 weeks. In embodiments, the active agent-containing layer(s) dissolves in about 5 days to about 1 week. In embodiments, the active agent-containing layer(s) dissolves in about 5 days to about 6 days. In embodiments, the active agent-containing layer(s) dissolves in about 6 days to about 1 month. In embodiments, the active agent-containing layer(s) dissolves in about 6 days to about 4 weeks. In embodiments, the active agent-containing layer(s) dissolves in about 6 days to about 3 weeks. In embodiments, the active agent-containing layer(s) dissolves in about 6 days to about 2 weeks. In embodiments, the active agent-containing layer(s) dissolves in about 6 days to about 1 week. In embodiments, the active agent-containing layer(s) dissolves in about 1 week to about 1 month. In embodiments, the active agent-containing layer(s) dissolves in about 1 week to about 4 weeks. In embodiments, the active agent-containing layer(s) dissolves in about 1 week to about 3 weeks. In embodiments, the active agent-containing layer(s) dissolves in about 1 week to about 2 weeks. In embodiments, the active agent-containing layer(s) dissolves in about 2 weeks to about I month. In embodiments, the active agent-containing layer(s) dissolves in about 2 weeks to about 4 weeks. In embodiments, the active agent-containing layer(s) dissolves in about 2 weeks to about 3 weeks. In embodiments, the active agent-containing layer(s) dissolves in about 3 weeks to about 1 month. In embodiments, the active agent-containing layer(s) dissolves in about 4 weeks to about 4 weeks. The dissolution time may be any value or subrange within the recited ranges, including endpoints. For example, the active agent-containing layer(s) may dissolve in about 24 hours, 30 hours, 48 hours, 3 days, 4 days, 5 days, 6 days, 7, 8, 9, 10, 11, 12, 13 days, 2 weeks, 3 weeks, 4 weeks, one month, etc. In various embodiments, the active agent-containing layer(s) may dissolve about 1, 2, 3, 4, 5, 6, 12, 18, 24 hours. In various embodiments, the active agent-containing layer(s) is dissolved in a solvent and lyophilized.

The PCL or co-polymer layers may be shaped or molded to adjust the size, shape, active agent elution rate, and/or dissolution rate of the patch or layer.

Patches

Topical patches include, but are not limited to, transdermal patches, as well as patches that can be applied to a mucosal membrane or wound of a subject. Transdermal patches may be applied, for example, to the skin of a subject. Further, patches as described herein may be applied internally to a subject, e.g., subdermal or within a body cavity.

A patch as described herein may contain one or more layers of soluble, hydrophilic PCL or co-polymer thereof. The patch may also contain one or more layers of an additional polymer. In embodiments, one or more layers of PCL and/or additional polymers contains an active agent. In embodiments, different layers contain different active agents. In embodiments, different layers contain the same active agent.

In embodiments, the patch includes a reservoir of active agent. Liquid reservoir patches of various designs are well known to researchers in the field of transdermal drug delivery. See, for example, U.S. Pat. Nos. 4,460,372 and 5,591,767, each of which is incorporated herein by reference in its entirety. In embodiments, a PCL or PCL co-polymer layer is adjacent to the reservoir. In embodiments, a PCL or PCL co-polymer layer is in fluid connection with the reservoir.

In embodiments, the patch comprises a film. The film may be positioned between the skin or other area of the subject and the active agent-containing layer. In embodiments, the film contains the first active agent or a different active agent. In embodiments, the film includes PCL or co-polymer thereof. In embodiments, the film includes modified PCL or co-polymer thereof. Film includes soluble, hydrophilic PCL or co-polymer thereof. In embodiments, the film acts as a vapor barrier between the subject's skin (or other area) and the active agent-containing layer(s). In embodiments, the film degrades over time after application of the patch to a subject.

In various embodiments, the patch may comprise one or more outer layers and one or more inner layers. The one or more outer layers may have a molecular weight of hydrophilic PCL between about 80,000 g/mol and 120,000 g/mol. The one or more outer layers may further have a weight/volume PCL concentration of about 5-8%. A thickness of the one or more outer layers may be about 100-300 μm. The one or more outer layers may be sandwiched within the patch. Alternatively, the one or more outer layers may be laminated into the patch. Active agents or other materials may be imbibed into one or more of the outer layers.

The one or more inner layers may have a molecular weight of hydrophilic PCL between about 20,000 g/mol and 80,000 g/mol. The one or more inner layers may further have a weight/volume PCL concentration of about 3-5%. Like the outer layers, the one or more inner layers may be sandwiched within the patch or laminated into the patch. A thickness of the one or more inner layers may be about 10-50 μm. The one or more inner layers may have a dimensional undersurface that is not flat. Active agents or other materials may be imbibed into one or more of the inner layers.

The patches may be configured to deliver various active agents. For example, the patches may be configured to deliver nutraceuticals or cosmeceuticals. The patches may be configured to deliver a medicinal or therapeutic agent. The patch may be configured to deliver a long term diagnostic that monitors infectious agents.

Microbeads

In various embodiments, the hydrophilic PCL may be prepared into microbeads of various sizes and properties. The microbeads may be prepared into creams or the like for treatment. In an exemplary embodiment, the microbeads may have a molecular weight of between 20,000 g/mol and 80,000 g/mol. The microbeads may have a weight/volume PCL concentration of about 3-5%. A diameter of the microbeads may be about 1-10 μm in length. In an exemplary embodiment, the microbeads may be lyophilized to remove solvents or other agents trapped within the microbeads.

In various embodiments, admixtures of conditioned stem cell media containing exosomes, cytokines, and/or growth factors may be produced with microbeads. Microbeads may be infused with active agents in various ways. For example, the microbeads may be coupled to an active agent through a peptide bond to an N-substituted terminal end of the PCL molecule. The infused microbeads may be mixed into skin creams and applied to the skin of a subject. In one example of a treatment with a microbead cream, stem cell infused microbeads may be applied to skin to fill rhytides (wrinkle crevices) and deliver various active agents through skin resorption over a period of time.

In an exemplary embodiment, the microbead admixtures may be configured to deliver therapeutic agents through nasal administration. Microbead admixtures that are configured for nasal administration may contain convalescent antiserum, passive immune agents including avian IgY antibodies which can neutralize viral & microbial pathogens or other therapeutic or medicinal agents. The microbeads may be produced with PCL with a molecular weight of about 20,000 g/mol and 80,000 g/mol, a PCL concentration of about 3-5% weight/volume, and a diameter of about 1-10 μm. In another exemplary embodiment, microbeads that are formulated with a passive-immune agent may be applied to a subject through intranasal aerosolization. The aerosolized microbeads may provide inactivation of viral or microbial pathogens to effect a short term immunity in the subject.

In yet another exemplary embodiment, the microbeads may be configured to self-monitor and detect pathogen infections. The microbeads may be produced with PCL with a molecular weight of about 80,000 g/mol and 120,000 g/mol, a PCL concentration of about 5-8% weight/volume, and a diameter of about 1-100 μm. The microbeads may be lyophilized. Reactive antigens or whole pathogens may be covalently linked to the microbeads. In various embodiments, the microbeads may be implanted into a subject via a 26-gauge syringe for real-time self-monitoring and detection of infection from pathogenic agents.

Gauze

In various embodiments, hydrophilic PCL may be formed into microfibers that are electrospun into a gauze to treat and heal wounds. The PCL used to form the microfibers may have a molecular weight of about 20,000 g/mol to 80,000 g/mol. The microfibers may have a weight/volume PCL concentration of 3-5%. A diameter of the microfibers may be 0.5-10 μm. The microfibers may be lyophilized. PCL that is electrospun into microfibers and woven may be used as a replacement for cotton gauze. In an exemplary embodiment, a gauze that is N-substituted to form a Diomat® PCL polymer may be imbibed with stem cell biologicals that are configured to heal wounds. Thus, unlike a cotton gauze, the Diomat® polymer gauze may be used as a first treatment. Further, the Diomat® gauze may be left on a wound and never actively removed. Instead, the Diomat® gauze may be resorbed into the body, along with healing agents, as the Diomat® polymer breaks down. The wound healing agents and stem cell derived biologicals may be lyophilized or copolymerized with the Diomat® polymer.

In various embodiments, PCL that is electrospun into microfibers that may be woven into a bandage under coverings. The PCL microfibers may be a Diomat® gauze that is imbibed with stem-cell biologicals that are tuned for wound and scar healing. In various embodiments, the Diomat® gauze may be a bandage insert that is never removed from a wound or burn. Instead, the biopolymer is resorbed into the body along with healing agents that were coupled to the Diomat® gauze. Thus, scabs may not be removed as the gauze is disintegrated. Further, PCL is an FDA approved polymer for surgical replacements. Wound-healing agents and stem cell derived biologicals may be lyophilized or co-polymerized with a Diomat® gauze and laminated onto an underside of standard bandages. In an exemplary embodiment, a Diomat® gauze may be wrapped around body parts after a tattooing process.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

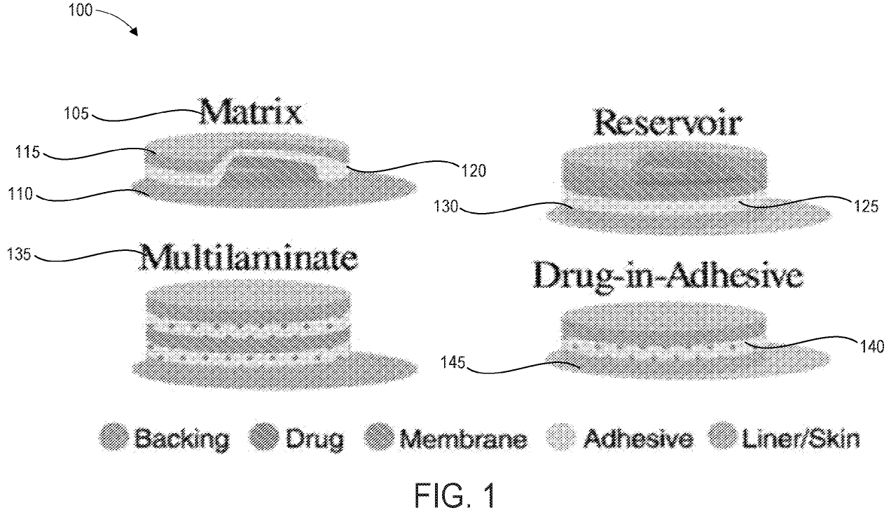

Referring to FIG. 1, FIG. 1 is an illustration 100 of examples of various embodiments of topical patches. In an exemplary embodiment, the topical patch may comprise a matrix of hydrophilic PCL. The hydrophilic PCL matrix 105 may be a material that is bound together by continuous fibers of hydrophilic PCL. An active agent may be coupled to the hydrophilic PCL matrix 105. After the application of the patch, the PCL matrix may slowly dissolve over time, which slowly releases the active agent. A film may comprise a liner/skin 110 that separates the polymer matrix from a target for adhesion. For instance, the liner/skin 110 may be attached to a wound to be treated. In another instance, the liner/skin 110 may be attached under the skin of a patient to release an antigen that diagnoses the presence of antibodies in the patient.

The PCL matrix may have a backing of material that provides support for the topical patch. The backing layer 115 is preferably made of a material or combination of materials that is substantially impermeable to the layer or layers with which it can be in contact, i.e., to the carrier layer and the active ingredient contained therein, the adhesives, etc. A primary objective is to prevent seepage of the active ingredient through the backing layer 115. The actual material used for the backing layer 115 will depend on the properties of the materials in contact therewith. Some suitable materials include, for example, cellophane, cellulose acetate, ethyl cellulose, plasticized vinyl acetate-vinyl chloride copolymers, ethylene-vinyl acetate copolymer, polyethylene terephthalate, nylon, polyethylene, polypropylene, polyvinylidene chloride (e.g., SARAN®), paper, cloth and aluminum foil. The material which forms this backing layer may be flexible or non-flexible. In various embodiments, a flexible backing layer is employed to conform to the shape of the body member to which the device is attached. It is to be understood that, when a patch is applied internally, a backing layer may not be needed. Thus, in embodiments, the patch does not include a backing layer.

In various embodiments, the patch includes an adhesive layer 120. The adhesive layer may include an active agent (e.g., the first, second, third, or a fourth active agent). In embodiments, the patch includes an active agent enhancer. The adhesive layer may be any adhesive that is appropriate for a topical or transdermal patch. Adhesives are well known in the art, including but not limited to those described in U.S. Pat. Nos. 5,948,433; 5,008,110; For example, any of the well-known dermatologically-acceptable, pressure-sensitive adhesives can be used as an adhesive. Example adhesives include, without limitation, silicones, polyisobutylene, and acrylic or methacrylic resins such as polymers of esters of acrylic or methacrylic acid with alcohols such as n-butanol, n-pentanol, isopentanol, 2-methyl butanol, I-methyl butanol, I-methyl pentanol, 2-methyl pentanol, 3-methyl pentanol, 2-ethyl butanol, isooctanol, n-decanol, or n-dodecanol, alone or copolymerized with ethylenically unsaturated monomers such as acrylic acid, methacrylic acid, acrylamide, methacrylamide, N-alkoxymethyl acrylamides, N-alkoxymethyl methacrylamides, N-tert-butylacrylamide, itaconic acid, vinylacetate, N-branched alkyl maleamic acids wherein the alkyl group has 10 to 24 carbon atoms, glycol diacrylates, or mixtures of these. Other examples of acceptable adhesives include those based on natural or synthetic rubbers such as silicone rubber, styrene-butadiene, butyl, neoprene, polybutadiene, polyisoprene, and polyurethane elastomers; vinyl polymers, such as polyvinylalcohol, polyvinyl ethers, polyvinyl pyrrolidone, and polyvinylacetate; cellulose derivatives such as ethyl cellulose, methyl cellulose, nitrocellulose, and carboxymethyl cellulose; and natural gums such as guar, acacia, karaya, pectins, starch, dextrin, albumin, gelatin, casein, etc. The adhesives may be compounded with tackifiers and stabilizers as is well known in the art. In embodiments, the adhesive adheres the patch to the skin or other area of the subject. It is to be understood that, when a patch is applied internally, to a wound, or to a mucosal membrane, an adhesive may not be needed and/or desired. Thus, in embodiments, the patch does not include an adhesive.

As shown in FIG. 1, the first active agent-containing layer may be a reservoir 125. In various embodiments, the first active agent-containing layer is adjacent to a membrane 130. In embodiments, the active agent is absorbed by the membrane 130. For example, the active agent may move from the reservoir 125, into the membrane 130, and then into the subject. The membrane 130 may act as a barrier to slow administration of the active agent to the subject. In embodiments, the membrane includes soluble, hydrophilic poly caprolactone. In embodiments, the membrane 130 contains a co-polymer of polycaprolactone (e.g., soluble, hydrophilic polycaprolactone) and a second polymer. In embodiments, the second polymer is polylactic acid, acrylamide, polylactide, polyglycolide, polydioxanone, poly N-isopropylacrylamide, polyurethane, a polyester other than PCL, a polystyrene, or a polyvinylidene. In embodiments, the membrane 130 is porous. In embodiments, the membrane 130 is a film.

In various embodiments, the topical patch may be assembled from multiple alternating layers 135 of various hydrophilic PCL materials that are coupled to an active agent. The various layers may be fixed together through various means. The hydrophilic PCL layers may be configured to dissolve at different rates. Additionally, the hydrophilic PCL layers may be coupled to identical or different active agents. In an example, a pain killing agent is coupled to a first PCL layer that dissolves quickly over a period 48 hours and an antibiotic agent is couple to a second PCL layer that dissolves more slowly over a period of 30 days.

As shown in FIG. 1, an active agent such as a drug may be combined with an adhesive. In an exemplary embodiment, a drug-in-adhesive layer 140 may be layered over a membrane 145. The membrane 145 may be configured to allow the drug to permeate through the membrane 145. The various layering embodiments shown in FIG. 1 are intended to illustrate examples of a few of the many possible embodiments of the disclosed subject matter when it is formed into a topical patch. More embodiments of the patch, such as with different combinations of layering, may be produced.

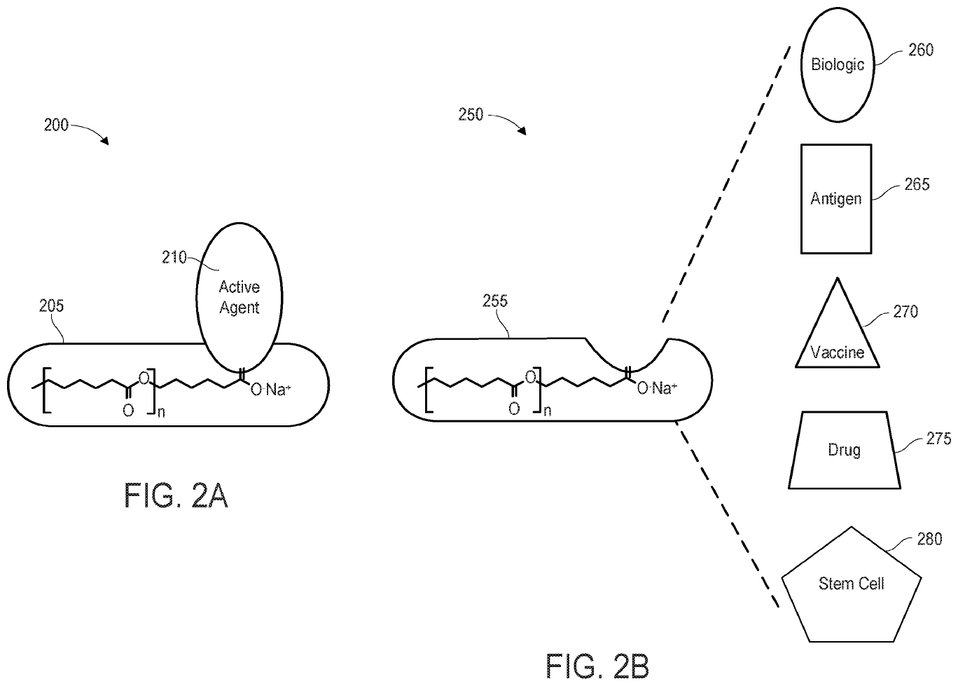

Referring to FIG. 2A, FIG. 2A is an illustration 200 of a chemical composition of a polycaprolactone molecule 205 that is covalently coupled to an active agent 210. In various embodiments, polycaprolactone may be coupled to an active agent 210. In an exemplary embodiment, the active agent couples to the polycaprolactone via a peptide bond after a carbonyl group of the polycaprolactone is acid catalyzed into a Schiff base. As shown in FIG. 2A, the active agent 210 is illustrated as an oval shape that couples to a carbonyl carbon of the polymer. The active agent 210 may be released as the polycaprolactone dissolves. In various embodiments, the polycaprolactone may be configured to dissolve responsive to contact with an aqueous solvent such as bodily fluids. The active agent 210 may be released at a rate that corresponds to the dissolution rate of the polycaprolactone.

Referring to FIG. 2B, FIG. 2B is an illustration 250 of a chemical composition of a polycaprolactone molecule 255 with an array of potential active agents for which the molecule may be bound. As shown, the potential active agent may take several potential forms. The active agent may be a biologic 260, an antigen 265, a vaccine 270, a drug 275, and a stem cell 280. In an example of a biologic 260, the active agent may be a protein that effectuates gene therapy in a patient. The biologic 260 may couple to the polycaprolactone molecule with various bond types. In one example, a biologic protein forms a peptide bond with the polycaprolactone molecule 255.

An example of an antigen 265 is a protein that creates an antibody response in a patient. For instance, the active agent may be an antigen 265 of SARS CoV2, which may be used as the active agent to test a patient for antibodies of COVID-19. A vaccine 270 may be an agent that stimulates the immune system of a patient to recognize that agent and produce an immune response. By attaching a vaccine 270 to a polycaprolactone material, the vaccine 270 may be released slowly into a patient rather than all at once. The slow release of the vaccine 270 may ease the immune response to the vaccine 270, thus reducing the immune stress that is delivered to the patient.

A drug 275 may be various chemicals or other substances that react with the body of a patient. For example, the drug 275 may be an immunosuppressant drug such as a steroid. The drug 275 may be coupled to the polycaprolactone material via a covalent or other type of bond.

In various embodiments, the polycaprolactone molecule may be coupled to stem cells 280. Stem cells 280 may proliferate for a period of time while maintaining an undifferentiated cell status. Daughter cells of the stem cells 280 may differentiate into various types of cells of host tissues, thus repairing the host tissue. The stem cells 280 may be released as the polycaprolactone molecule dissolves. Stem cells 280 may have a variety of applications. It has been shown that application of stem cells 280 may positively affect the expression of inflammatory factors involved in wound healing. Stem cells 280 may improve wound healing in diabetic patients. Additionally, stem cells 280 may stimulate cellular growth. A topical treatment of stem cells 280 may stimulate regeneration of cells to decrease wrinkles or otherwise positively affect the biomechanical parameters of the skin or central nervous system.

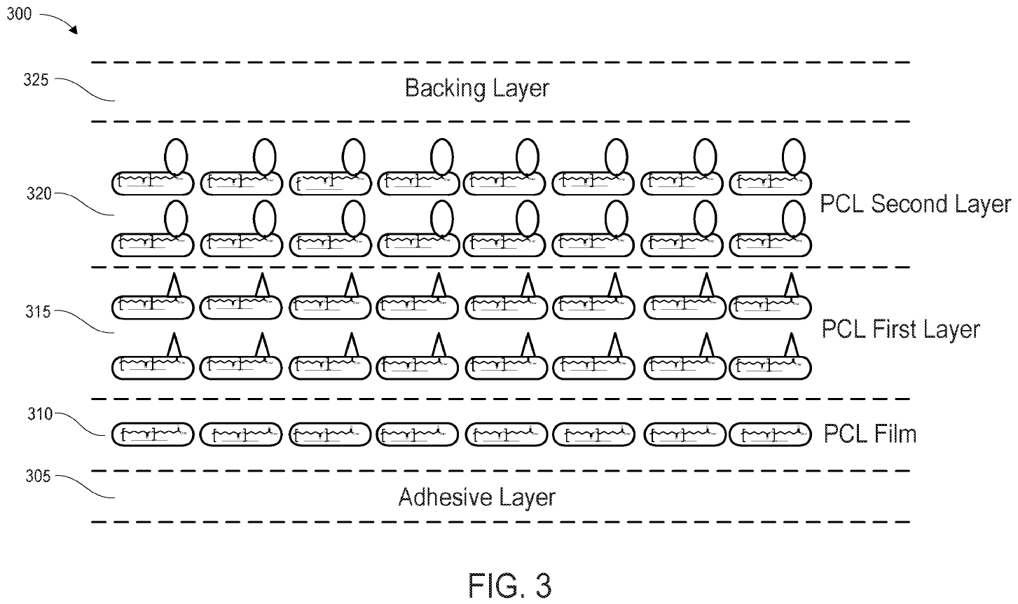

Referring to FIG. 3, FIG. 3 is an illustration of various polycaprolactone layers of a medical device that may release an active agent into a subject. The medical device may comprise a patch or other form factors of polycaprolactone. The medical device may include an adhesive layer 305, a polycaprolactone film 310, a polycaprolactone first active layer 315, a polycaprolactone second active layer 320, and a backing layer 325.

The adhesive layer 305 may adhere the medical device to a subject. For instance, the adhesive layer 305 may comprise a biodegradable adhesive that fixes the medical device to the subject. The polycaprolactone film 310 may act as a barrier between the polycaprolactone first active layer 315 and the subject. A rate of dissolution of the polycaprolactone film 310 may be configured based on the treatment requirements of the subject. For instance, a polycaprolactone film 310 may be configured to dissolve in 1-3 hours after the medical device has been applied to the subject.

The polycaprolactone first active layer 315 may comprise polycaprolactone or a copolymer of polycaprolactone that is coupled to an active agent. The active agent may be various substances that effectuate a reaction in the subject. Similarly, the polycaprolactone second active layer 320 may also comprise polycaprolactone or a copolymer of polycaprolactone that is coupled to an active agent. The polycaprolactone second active layer 320 may have a different dissolution rate, dissolution time, or different active agent from the polycaprolactone first active layer 315. For example, the polycaprolactone second active layer 320 may have the same dissolution rate as the polycaprolactone first active layer 315, but with a different active agent. In another example, the poly caprolactone second active layer 320 may have the same rate of dissolution but be thicker and thus dissolve over a longer period of time.

The backing layer 325 may provide support for the medical device. Further, the backing layer 325 may provide protection from outside elements. For instance, the backing layer 325 may comprise an impermeable material that prevents liquids from leaking through to the polycaprolactone layers.

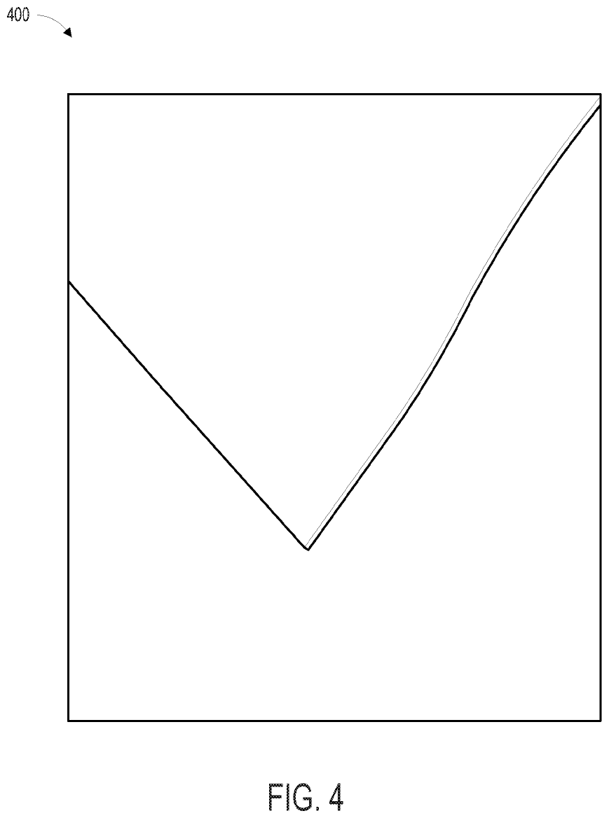

Referring to FIG. 4, FIG. 4 is a photograph 400 of an embodiment of Diomat® film. The Diomat® film may comprise hydrophilic polycaprolactone that has been N-substituted to create a Schiff base. The Diomat® film may be treated to control the dissolution rate of the Diomat® film. For instance, the amount of time that the Diomat® film is treated with a base may control the dissolution rate of the Diomat® film. Further, the molecular weight of the polycaprolactone may control the dissolution rate of the Diomat® film. The dissolution rate may increase proportionately as the molecular weight is decreased.

The Diomat® film may be prepared by dissolving 4 g of PCL pellets in 40 mL of methylene chloride. The polymer solution may be cast onto a glass substrate and the solvent removed by controlled evaporation at room temperature over a period of 24 hr. The pristine PCL film with a thickness of 40-100 um is washed with copious amounts of alcohol/water (1/1, v/v) solution (pH 12) for a pre-determined period at 37 C to produce the hydrolyzed PCL-OH film. The dried PCL-OH film may be cut into various specimen sizes.

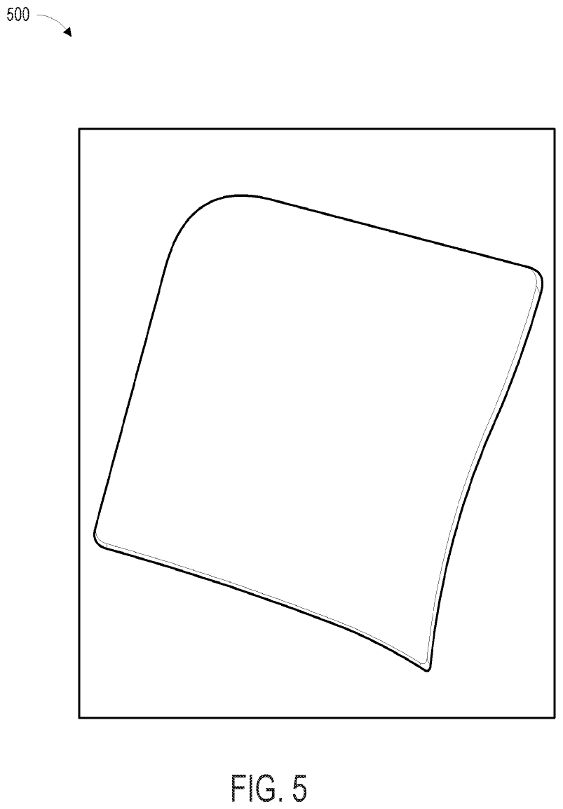

Referring to FIG. 5, FIG. 5 is a photograph 500 of an embodiment of Diomat® foam. The Diomat® foam may be coupled to an active agent. The Diomat® foam may further comprise hydrophilic poly caprolactone that has been N-substituted to create a Schiff base. The Diomat® foam may be affixed to a subject to administer the active agent to the subject. The Diomat® foam may comprise additional layers based on the desired treatment. For example, the Diomat® foam may be layered within an adhesive and a backing.

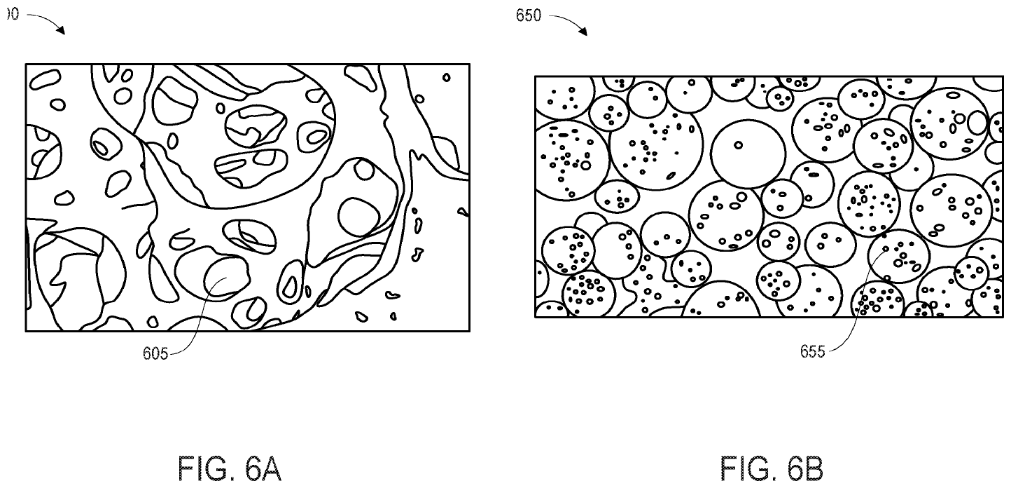

Referring to FIG. 6A, FIG. 6A is an electron microscopy image showing the microporous structure of the base-treated Diomat® foam. The structural components of the solid phase of poly caprolactone matrix, namely the porosity of the may appear to have a somewhat non-laminar configuration as though some were cut from a single sheet, it will be understood that this appearance may in part be attributed to the difficulties of representing complex three-dimensional structures in a two dimensional figure.

The PCL foam may comprise hydrophilic polycaprolactone that has been N-substituted to create a Schiff base. The size and number of holes 605 in the PCL foam may correspond to a porosity of the PCL foam. Porosity may be inversely proportional to the dissolution rate of the PCL foam. The porosity has been found to be proportional to the molecular weight and weight per volume of the PCL foam. Thus, to increase the dissolution rate, a polycaprolactone with a lower molecular weight and/or lower weight per volume may be used to produce the PCL foam.

Referring to FIG. 6B, is an electron microscopy image showing the microporous structure of PCL microbeads. Like the PCL foam, the PCL microbeads may comprise hydrophilic polycaprolactone that has been N-substituted to create a Schiff base. And like the PCL foam, the size and number of holes 655 in the PCL microbeads may correspond to a porosity of the PCL microbeads. The microbeads may be coupled with an active agent. The active agent may be delivered to a subject as the PCL microbeads dissolve. In an exemplary embodiment, the active agent may be covalently bonded to the PCL microbeads.

PCL microbeads may be prepared by stirring polycaprolactone in a solvent at a high rate such as 6000 rpm for about 2 minutes. The microbeads, thus formed, may be isolated by centrifugation. PCL microbeads may be washed and dried. For the preparation of PCL nanospheres of smaller diameter, the above procedure may be modified by increasing the stir rate and time. For example, a stirring speed of 12000 rpm for 5 minutes may result in Diomat® nanospheres.

The PCL microbeads may be used as the polycaprolactone active layer in a topical patch. The thickness of the patch may be controlled by increasing or reducing the number of PCL microbeads. The dissolution rate of the PCL microbeads may be controlled in one or more ways. For instance, the time of treatment with a base of pH 8 or greater may be proportional to the dissolution rate. Further, the molecular weight and weight per volume of polycaprolactone used to produce the PCL microbeads may be inversely proportional to the dissolution rate. The thickness of a layer may increase the time of dissolution.

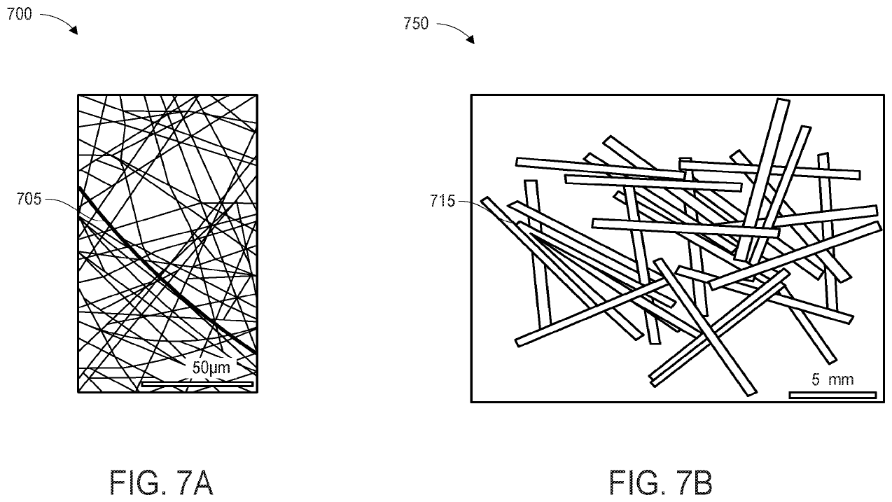

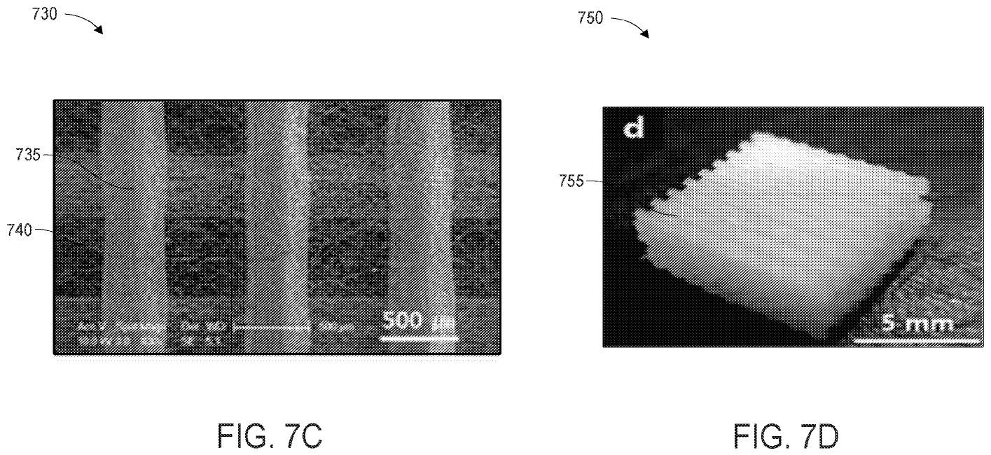

Referring to FIG. 7A, FIG. 7A is a magnified photograph 700 of an embodiment of polycaprolactone fibers 705 that are produced from electrospinning. Like microbeads, the polycaprolactone fibers 705 may be N-substituted to create a Schiff base that can be coupled to an active agent. The polycaprolactone fibers 705 may be woven into various structures. For example, the polycaprolactone fibers 705 may be woven into a gauze that can be dressed onto a wound.

Referring to FIG. 7B, FIG. 7B is a magnified photograph 710 of an embodiment of polycaprolactone rods 715 that may be bound in a microstructure. In various embodiments, the polycaprolactone rods may be stacked to produce a structure that may be implanted into a subject. As shown in FIG. 7C, the polycaprolactone rods 735 are stacked and bound together with polycaprolactone fibers 740 that are wrapped around the polycaprolactone rods 735. The polycaprolactone rods 735 and/or fibers may be configured to dissolve over a period of time. Further, the polycaprolactone rods 735 and/or polycaprolactone fibers 740 may be coupled to an active agent. The active agent may be delivered to a subject as the polycaprolactone rods 735 and/or polycaprolactone fibers 740 dissolve.

Electro-spinning may be used to form the poly caprolactone fibers 705 as they are wrapped around the polycaprolactone rods 715. The electro-spun polycaprolactone fibers 705 may be prepared with a diameter a tens of nanometers to several microns. The polycaprolactone rods 715, bound by electro-spun polycaprolactone fibers 705 may be built into larger microstructures such as scaffolds.

Referring to FIG. 7D, FIG. 7D is a magnified photograph 750 of a microstructure 755 that is constructed from polycaprolactone rods 735 that are bound by polycaprolactone fibers 740. In various embodiments, the microstructure may form a scaffold from which treatment structures may be placed.

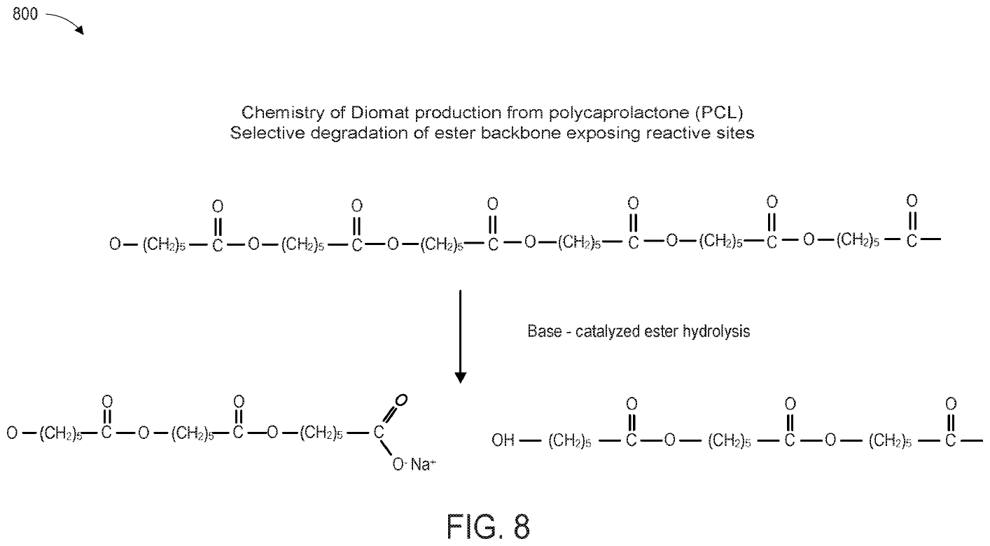

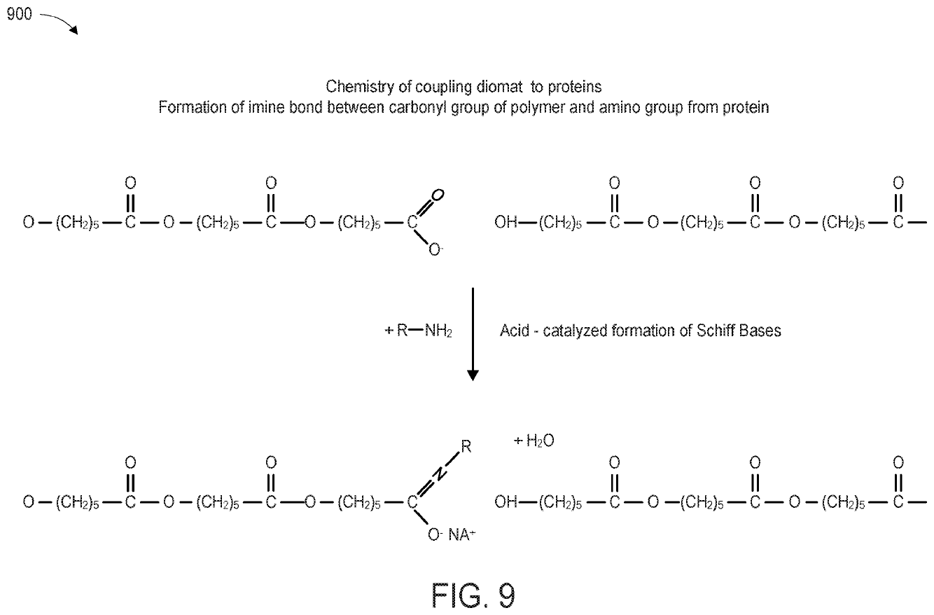

Referring to FIG. 8, FIG. 8 is a reaction diagram 800 of base-catalyzed hydrolysis of the ester linkages present in the backbone of polycaprolactone. The preparation of the Diomat® material is shown in FIGS. 8 and 9. The Diomat® material may be prepared by a base-catalyzed hydrolysis of the ester linkages present in the backbone of poly caprolactone. When treated with base, the reaction disrupts the polymer by creating carbonyl group on one side of the disrupted polymer and a hydroxyl group on the other side of the polymer. The time of treatment with the base may be correlated to the dissolution rate of the Diomat® material.

Referring to FIG. 9, FIG. 9 is a reaction diagram 900 of a coupling of surface-exposed carbonyl groups to create a Schiff base. The surface-exposed carbonyl groups, the production of which is shown in FIG. 8, can be covalently coupled to the amino groups present on the termini of basic amino acid residues found in most all proteins. The result is creation of a very stable Schiff base (C═N), thus serving to sequester an active agent to the Diomat polymer. In various embodiments, the active agent may be a protein. Also, in various embodiments, the active agent may be an organic molecule that bound to the nitrogen of the Schiff base.

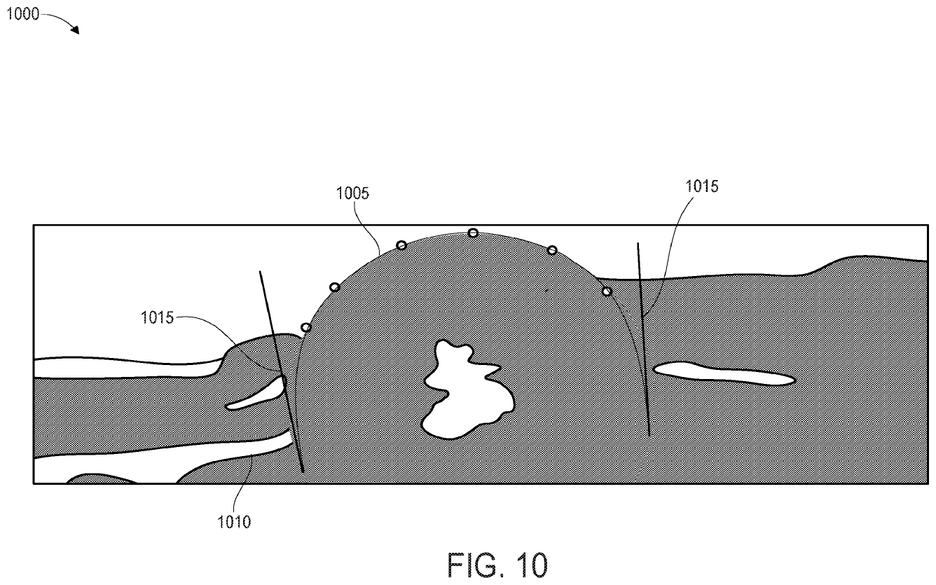

Referring to FIG. 10, FIG. 10 is a magnified photograph of a hydrophobicity test of a water droplet 1005 on a polycaprolactone wafer 1010. The hydrophilicity of polycaprolactone may be determined by observing the interaction of a flat polycaprolactone wafer 1010 with a droplet of a polar liquid such as water. The contact angle, which is the angle that the sides of the water droplet 1005 make with the plane of the polycaprolactone wafer 1010, is indicative of the hydrophobicity of the surface of the polycaprolactone wafer 1010.

A low angle (<900) indicates that the material is hydrophilic while a higher angle (>90°) indicates that the material is hydrophobic. A hydrophobicity test was conducted on multiple polycaprolactone samples that were treated in various ways to control and modify the hydrophobicity of the samples. The contact angle, as indicated by the angle of the tangent lines 1015, that are drawn on either side of the droplet, with the plane of the wafer, is approximately 720, thus indicating that the wafer is hydrophilic.

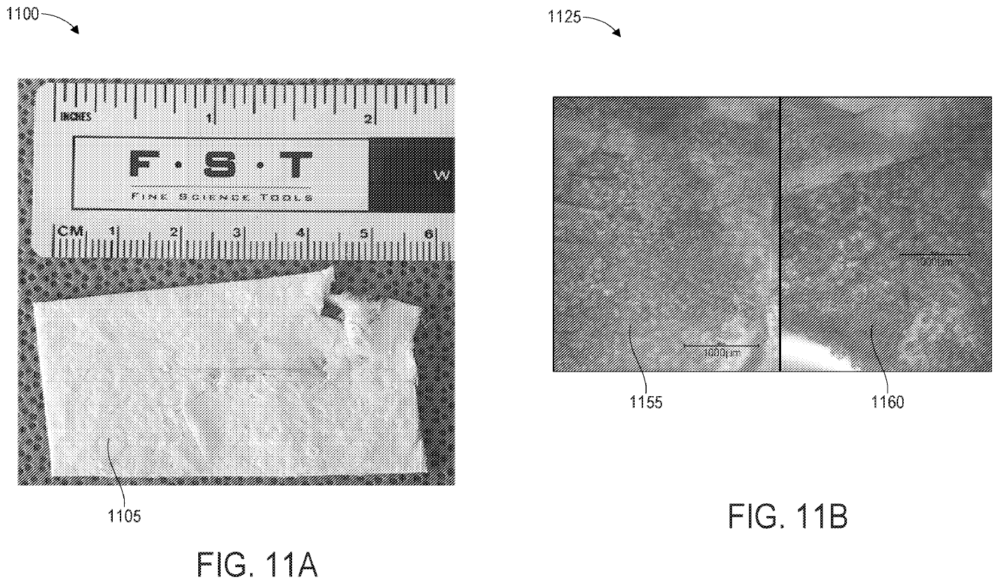

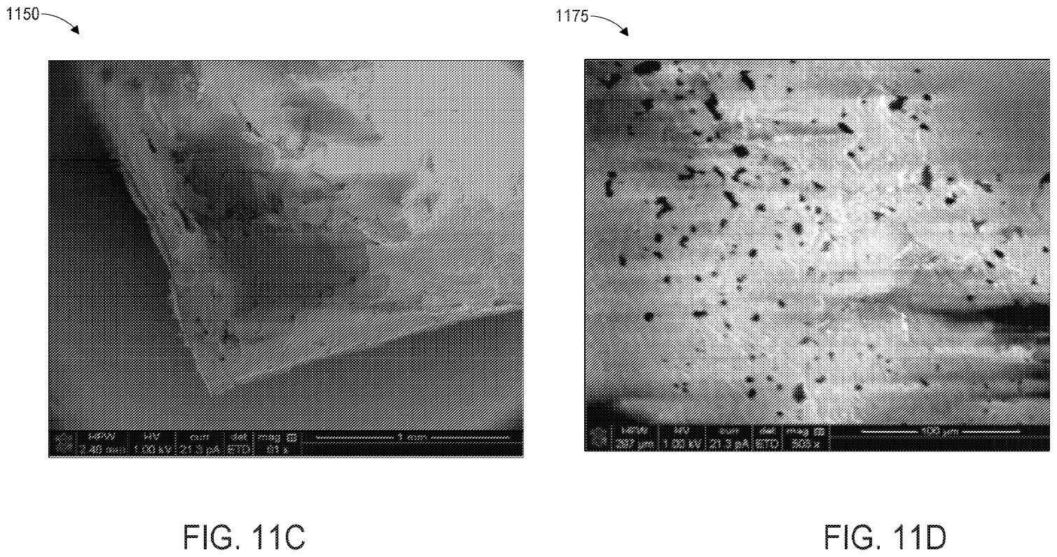

Various hydrophobicity tests were performed on polycaprolactone samples. Parameters such as molecular weight, weight/volume, and sodium hydroxide treatment were tested. Images of various polycaprolactone samples are shown in FIGS. 11A-14D. A porosity and smoothness of the polycaprolactone samples may be visible in the figures.

TABLE 1

| | Molecular Weight | Thickness | Base |
|---|---|---|---|
| Sample | (kg/mol) | (mm) | Treatment |
| S1 | 45 | 0.5-1 | + |
| S2 | 45 | 0.5-1 | − |
| S3 | 45 | 0.5-1 | + |
| S4 | 93 | 0.8 | − |

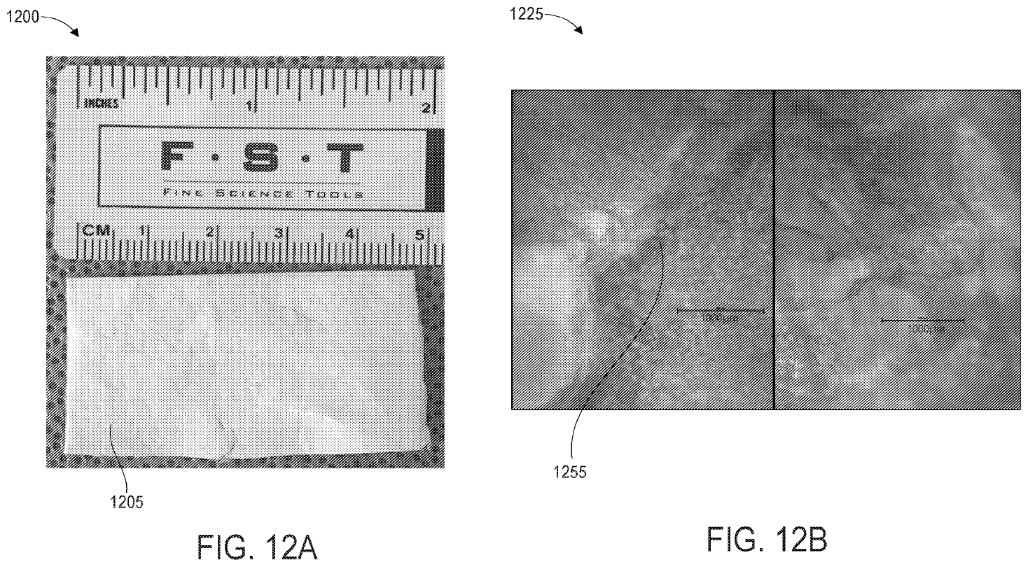

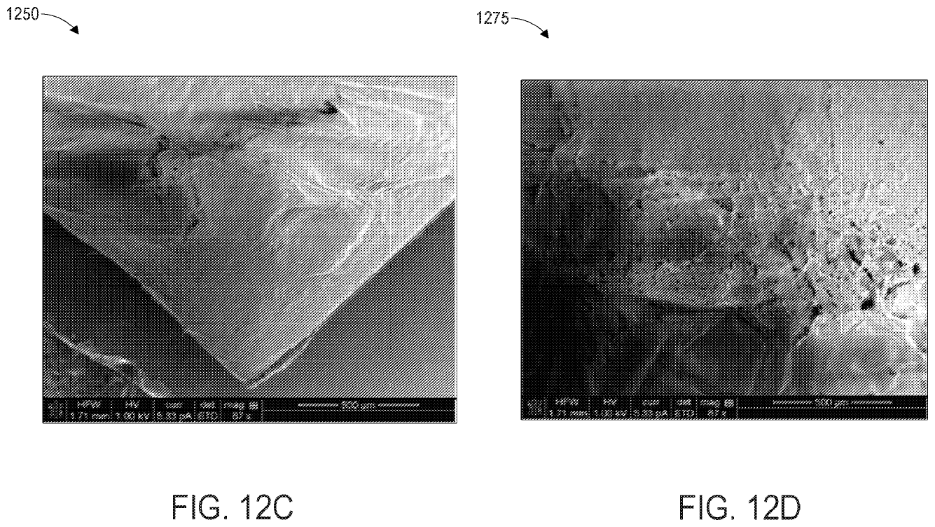

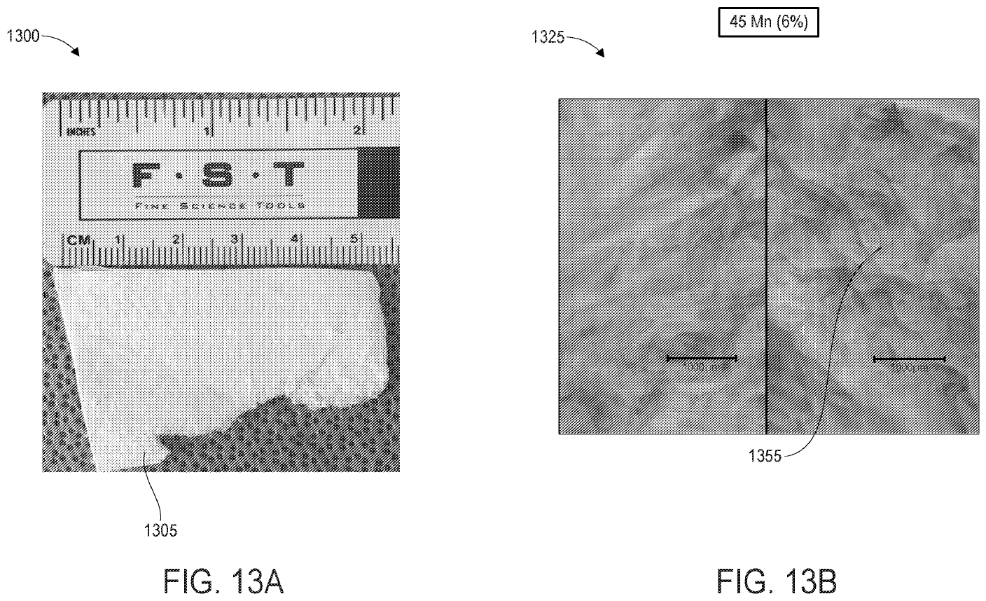

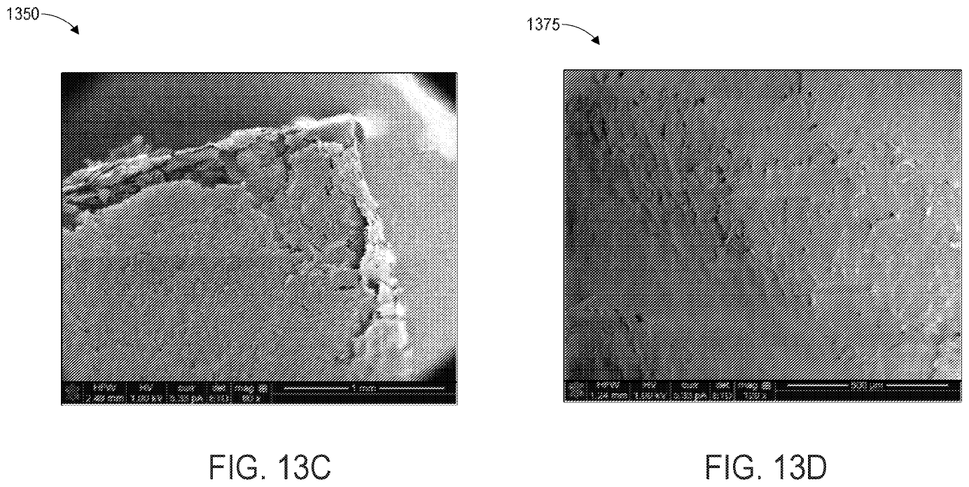

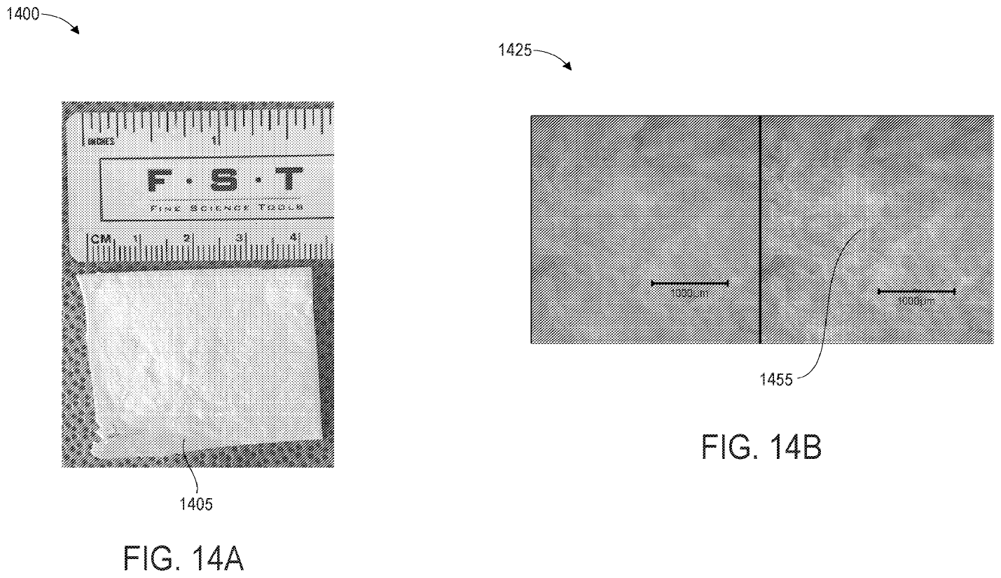

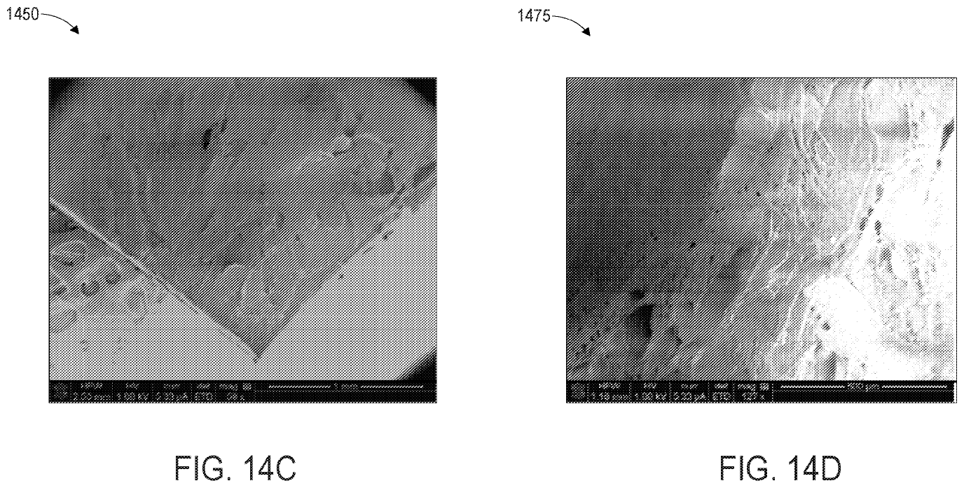

Table 1 shows the parameters of samples 1-4, which are displayed in FIGS. 11A-14D. Sample S1 is shown in FIGS. 11A-11D. Sample S2 is shown in FIGS. 12A-12D. Sample S3 is shown in FIGS. 13A-13D. Sample S4 is shown in FIGS. 14A-14D.

Referring to FIG. 11A, FIG. 11A is an image 1100 of a full Diomat® sheet 1105 sample S1. The sample shown in the image 1100 was made using 5% 45 mw (Sigma) with a 0.5-1.0 mm followed by NaOH. Notice the higher surface porosity under inverted light microscopy. The full Diomat® sheet 1105 was purchased from Sigma before treatment. It has a thickness of 0.1-0.5 mm. The full Diomat® sheet 1105 shown in FIG. 11A was treated with sodium hydroxide.

Referring to FIG. 11B, FIG. 11B is an inverted brightfield microscopy image 1125 of Diomat sample S1 using 4× objective. The scale bar of the image 1125 is 500 μm. This sample was made using 5% 45 mw (Sigma) with a 0.5-1.0 mm followed by NaOH treatment. Notice the surface porosity under inverted light microscopy. The pore size range, which is dependent on lyophilization conditions, is 10 μm-5000 μm in diameter. Examples of pores are pore 1155 and pore 1160.

Referring to FIG. 11C, FIG. 11C is an electron microscopy image 1150 of side-profile of Diomat® sample S1 using 1 mm (61×) resolution. This sample was made using 5% 45 mw (Sigma) with a 0.5-1.0 mm followed by NaOH. It was found that the smoothness of the full Diomat® sheet 1105 was inversely proportional to the hydrophilicity of the sample. The same sample is shown in FIG. 11D, which is a is an electron microscopy image 1175 of side-profile of Diomat® sample S1 using 1 mm (503×) resolution. The scale bar=1000 μm. This sample was made using 5% 45 mw (Sigma) with a 0.5-1.0 mm followed by NaOH.

Referring to FIG. 12A, FIG. 12A is an image 1200 of a full Diomat® sheet 1205 sample S2. This sample was made using 5% 45 mw (Sigma) with a 0.5-1.0 mm with no base treatment. Notice the higher surface porosity under inverted light microscopy. FIG. 12B is an inverted brightfield microscopy image 1225 of full Diomat® sample S2 using 4× objective. The scale bar=1000 μm. This sample was made using 5% 45 mw (Sigma) with a 0.5-1.0 mm with no base treatment. Notice the higher surface porosity under inverted light microscopy. The pore size range, which is dependent on lyophilization conditions, is 10 μm-5000 μm in diameter. An example of a pore is pore 1255.

Referring to FIG. 12C, FIG. 12C is an electron microscopy image 1250 of side-profile of Diomat® using 0.5 mm (87×) resolution. The scale bar=1000 μm. This sample was made using 5% 45 mw (Sigma) with a 0.5-1.0 mm with no base treatment. Likewise, FIG. 12D is an electron microscopy image of side-profile of Diomat® using 0.5 mm (87×) resolution. Scale bar=1000 μm. This sample was made using 5% 45 mw (Sigma) with a 0.5-1.0 mm with no base treatment. The lack of NaOH treatment for the full Diomat® sheet 1205 shown in FIG. 12A results in polycaprolactone molecules that are unbroken and thus have a lower hydrophilicity than samples that have been treated with NaOH. The length of time that samples are treated in NaOH may be adjusted to control to dissolution rate.

Referring to FIG. 13A, FIG. 13A is an image 1300 of a full Diomat® sheet 1305 sample S3. This sample was made using 6% 45 mw (Sigma) with a 0.5-1.0 mm with base treatment. Notice the lower surface porosity under inverted light microscopy. FIG. 13B is an inverted brightfield microscopy image 1325 of Diomat® sample S3 using 4× objective. The scale bar=1000 μm. This sample was made using 6% 45 mw (Sigma) with a 0.5-1.0 mm with base treatment. Notice the lower surface porosity under inverted light microscopy. The pore size range, which is dependent on lyophilization conditions, is 10 μm-5000 μm in diameter. An example of a pore is pore 1355.

Referring to FIG. 13C, FIG. 13C is an electron microscopy image 1350 of side-profile of Diomat® using 0.5 mm (87×) resolution. The scale bar=1000 μm. This sample was made using 6% 45 mw (Sigma) with a 0.5-1.0 mm with base treatment. Similarly, FIG. 13D is an electron microscopy image 1375 of side-profile of Diomat® using 0.5 mm (87×) resolution. Scale bar=1000 μm. This sample was made using 6% weight/volume 45 mw (Sigma) with a 0.5-1.0 mm with base treatment. The sample shown in FIG. 13A has a higher weight/volume than the samples shown in FIGS. 11A, 12A, and 14A of 6% vs. 5%. Based on the weight/volume parameter, sample S3 shown in FIG. 13A may be less porous and have a slower dissolution rate than the other samples.

Referring to FIG. 14A, FIG. 14A is an image 1400 of a full Diomat® sheet 1405 sample S4. This sample was made using 5% 93 mw (Sigma) with a 0.5-1.0 mm with no base treatment. Notice the lower surface porosity under inverted light microscopy. FIG. 14B is an inverted brightfield microscopy image 1425 of Diomat® sample S4 using 4× objective. The scale bar=1000 μm. This sample was made using 5% 93 mw (Sigma) with a 0.5-1.0 mm with no base treatment. Notice the lower surface porosity under inverted light microscopy. The pore size range, which is dependent on lyophilization conditions, is 10 μm-5000 μm in diameter. An example of a pore is pore 1455.

Referring to FIG. 14C, FIG. 14C is an electron microscopy image 1450 of side-profile of Diomat® using 0.5 mm (59×) resolution. The scale bar=1000 μm. This sample was made using 5% 93 mw (Sigma) with a 0.5-1.0 mm with no base treatment. The molecular weight of the sample shown in FIGS. 14A-14D, which is higher than the samples shown in FIGS. 11A-13D, is a factor that indicates a slower dissolution rate. Thus, samples with varying molecular weight may be used to tune the rate of dissolution of the polycaprolactone or copolymerized polycaprolactone with or without an active agent. FIG. 14D is an electron microscopy image 1475 of side-profile of Diomat® using 0.5 mm (127×) resolution. The scale bar=1000 μm. This sample was made using 5% 93 mw (Sigma) with a 0.5-1.0 mm with no base treatment.

1-74. (canceled)

75. A treatment substance, the treatment substance comprising:
microbeads comprising polycaprolactone that is infused with an active agent; and
wherein the microbeads are configured to be applied to a subject via nasal administration.

76. The treatment substance of claim 75, wherein the microbeads have a diameter of about 1-10 μm.

77. The treatment substance of claim 75, wherein the polycaprolactone has a molecular weight of about 20,000 g/mol-80,000 g/mol.

78. The treatment substance of claim 75, wherein the microbeads are lyophilized.

79. The treatment substance of claim 75, wherein the microbeads are mixed in a skin cream.

80. The treatment substance of claim 75, wherein the microbeads are configured to be applied via intranasal aerosolization.

81. The treatment substance of claim 75, wherein the active agent is an antigen or a whole pathogen.

82. A topical treatment medium, the topical treatment medium comprising:
a polyester material that has been treated with a base having a pH greater than 8 and a neutralizing agent for increasing hydrophilicity; and
the polyester material coupled to an active agent; and
wherein the polyester material comprises microbeads that are configured to be applied to a subject via nasal administration.

83. The topical treatment medium of claim 82, wherein the microbeads have a diameter of about 1-10 μm.

84. The topical treatment medium of claim 83, wherein the polyester material has a molecular weight of about 20,000 g/mol-80,000 g/mol.

85. The topical treatment medium of claim 83, wherein the microbeads are lyophilized.

86. The topical treatment medium of claim 83, wherein the microbeads are mixed in a skin cream.

87. The topical treatment medium of claim 83, wherein the microbeads are configured to be applied via intranasal aerosolization.

88. The topical treatment medium of claim 83, wherein the active agent is an antigen or a whole pathogen.

89. A method for delivering a therapeutic agent to a subject, the method comprising:
treating a polyester material with a with a base having a pH greater than 8 and a neutralizing agent for increasing hydrophilicity;
infusing the polyester material with an active agent;
delivery the polyester material to the subject through nasal administration; and
wherein the polyester material comprises microbeads.

90. The method of claim 89, wherein the polyester material comprises polycaprolactone.

91. The method of claim 89, wherein the polyester material comprises a copolymer of polycaprolactone.

92. The method of claim 89, wherein the microbeads have a diameter of about 1-10 μm; and
wherein the polyester material has a molecular weight of about 20,000 g/mol-80,000 g/mol.

93. The method of claim 92, wherein the polyester material is configured to dissolve between about 48 hours to about 72 hours.

94. The method of claim 92, wherein the microbeads are configured to be applied via intranasal aerosolization.
